(19) United States
(12) Patent Application Publication
(10) Pub. No.: US 2009/0038626 A1
(43) Pub. Date: Feb. 12, 2009

(54) Refractive vision correction for preventing presbyopia

(76) Inventor: Iku Kuo, Thaipao City (TW)

Correspondence Address: KAMRATH & ASSOCIATES P.A., 4825 OLSON MEMORIAL HIGHWAY, SUITE 245, GOLDEN VALLEY, MN, 55422, US

(21) Appl. No.: 11/836,272

(22) Filed: Aug. 9, 2007

(51) Int. Cl. A61F 9/013

(52) U.S. Cl. 128/898; 606/5

(57) ABSTRACT

The present invention relates to a refractive vision correction for preventing presbyopia; in particular, to the common refractive vision correction of dealing with myopia, hyperopia or astigmatism such as the problems of seeing for far distance objects, which is applied to and within a corresponding area of the center of the cornea when the pupil is in a size which is under normal light condition, and furthermore, other circular wavy corrections are orderly applied in accordance with different presbyopia degree to the scope beyond said corresponding area and overlapped by another corresponding area of the cornea when the pupil is in its most dilation; therefore, the present invention of pre-performing the circular wavy corrections can provide the patient with plural number of different curves of correction for preventing presbyopia when the patient get older with failing eyesight. The different curves of correction can appropriately improve and remedy the vision for the patient to see nearby objects when in different age with different presbyopia degree and achieve the expected goal of preventing presbyopia.

### FIELD OF THE INVENTION

The present invention provides a preventing method of refractive vision correction to presbyopia which is applying different curve of corrections according to different areas of the cornea; the method of correction can not only solve vision problem to the patient at that time and can also improve the failing eyesight as presbyopia in the future.

### BACKGROUND OF THE INVENTION

As shown in FIG. 1, the eye functions is normally by the cornea 2 to twist the coming light source to get through the pupil 3, and then the iris 5 works to adjust the light source by expanding or reducing the pupil 3, so that the light source can pass the lens 4 of the eyeball 1 and to be refracted and focused on the retina 6 in order to form an image, after that, the image is delivered to the cerebrum by the optic nerve 7, in which the shape of the cornea 2 is very important for it is in charge of at lest two thirds focusing function of the eyeball 1, the normal function of the cornea 2 can twist the light source accurately and clearly focus the image on the retina 6, on the contrary, the vision is blurred since the cornea 2 or the lens 4 can not twist the light source accurately.

For example, myopia is caused when the light come into the eye and is focused in front of the retina 6 as a result to see things not clear in a far distance place as shown in FIG. 2, and when if the light source is focused behind the retina 6, the hyperopia is caused as shown in FIG. 3; in addition, the result of astigmatism is because the uneven spherical curvature of the cornea 2 so as to cause the unfocused image as shown in FIG. 4. Further, the presbyopia is the problem of decaying, aging, decreasing of the lens flexibility or bad traction of ciliary body, therefore causing poor function of focusing adjustment of the eyeball, especially to see nearby things blurred; nevertheless, when the patient of myopia grows old, the eye function is aging as well, therefore, the patient will still encounter with same problem of presbyopia.

The above diseases are caused by abnormal focusing of eyes and so called refractive errors, the refractive errors can also be seen as irregular shape of the eyeball or the lens 4 with unusual function. Except for the traditional correction of wearing regular glasses or contacts, the Excimer Laser is currently a surgery to improve the ability of focusing of the eyeball; that is by using laser beam to gasify a thin layer tissue 20 to the center of the cornea 2, in order to even the radian of the center of the cornea 2 in order to achieve the goal of myopia correction as shown in FIG. 5. The surgery can correct the degree from 100 to 2000 with highly accuracy and the eyeball can still stay strong without side effect of nyctalopia and glare so as to greatly improve the safety and accuracy of the correction, furthermore, the surgery can also do the correction to hyperopia and astigmatism.

Although after the laser surgery the degree can be reduced and the patient can live without wearing glasses for about 5 to 10 years, the adjustment function of the eyeball is still failing for the patient getting older and the presbyopia degree will then be increased year after year and so as the hyperopia and astigmatism.

### SUMMARY OF THE INVENTION

Due to overcome the abovementioned problem if the patient has presbyopia after the correction of the surgery, the method of using by the present invention is to perform a current refractive vision correction seeing far distance objects to the corresponding area of the center of the cornea when the pupil is in a normal light condition, and furthermore, performing other circular wavy corrections to a predetermined scope which is around and beyond the center of the cornea continuously and orderly with a decided curve from inside-out.

Therefore, after said circular wavy corrections with the decided curve performed to the predetermined scope of the cornea inside-out, the eyesight of the patient can be corrected at that time and when the patient has presbyopia in the future, the pre-performing circular wavy corrections can also help to improve the eyesight for the patient to see nearby objects, therefore the goal of eyesight correction and prevention can be achieved in advance.

The present invention will become more fully understood from the detailed description given herein below and the accompanying drawings which are given by way of illustration only, and thus are not limitative of the present invention, and wherein:

### BRIEF DESCRIPTION OF THE DRAWINGS

FIG. 1 is a schematic view of normal vision.

FIG. 2 is a schematic view of myopia.

FIG. 3 is a schematic view of hyperopia.

FIG. 4 is a schematic view of astigmatism.

FIG. 5 is a schematic view of prior art after performing laser surgery.

FIG. 6 is a schematic view after receiving myopia surgery according to the present invention.

FIG. 7 is an enlarged schematic view of the cornea after receiving myopia surgery according to the present invention.

FIG. 8 is a schematic view after receiving hyperophia surgery according to the present invention.

FIG. 9 is an enlarged schematic view of the cornea after receiving hyperophia according to the present invention.

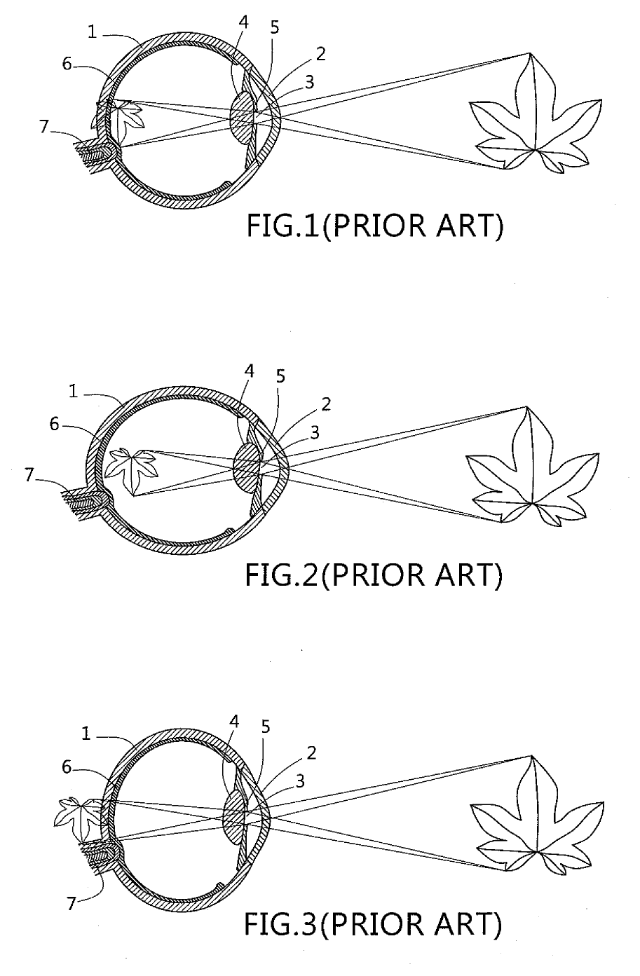

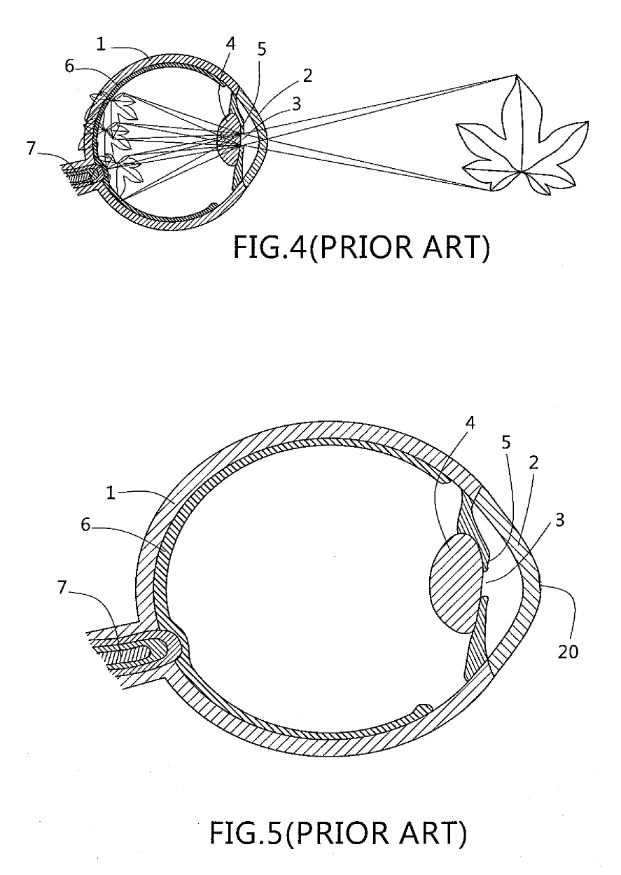

### DETAILED DESCRIPTION OF THE INVENTION

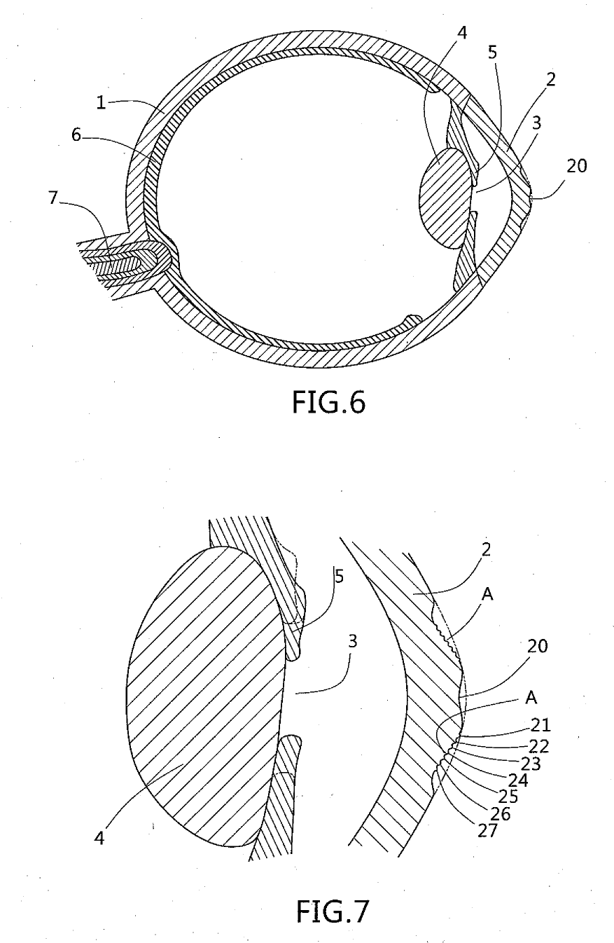

Referring to FIGS. 6 and 7, if the patient has myopia, that is because the patient has excess length of the eyeball axle or the cornea and the lens-bending properties are too strong, so the first step is to perform a refractive vision correction 20 with a layer of predetermined curve upon a corresponding area of the center of the cornea 2 when the pupil 3 is in the size of a normal light condition, furthermore, to form a concave surface A by a decided curve outward from the corresponding area of cornea, the last is to perform the convex of circular wavy corrections 21, 22, 23 . . . 27 continuously and orderly from inside out upon the concave surface A. Each of the corrections would project the images in different positions in front of the retina, such as the refractive vision correction 20 in the center area is conformed to the requirement which deals with actual myopia degree at that time for the patient, and as for the convex of circular wavy corrections of 21, 22, 23 . . . 27 are under the requirement for preventing future presbyopia and improving patient's vision of seeing nearby objects in a good condition, for example, when the patient is under age 50 and having slight presbyopia of seeing nearby objects with a blurred version, the circular wavy correction 21 can just correct the slight presbyopia for projecting the image on the retina properly, therefore, the clearness of the vision can be improved by the correction; as for the other corrections of 22, 23 . . . 27 can also properly for different age to improve vision in order to avoid presbyopia.

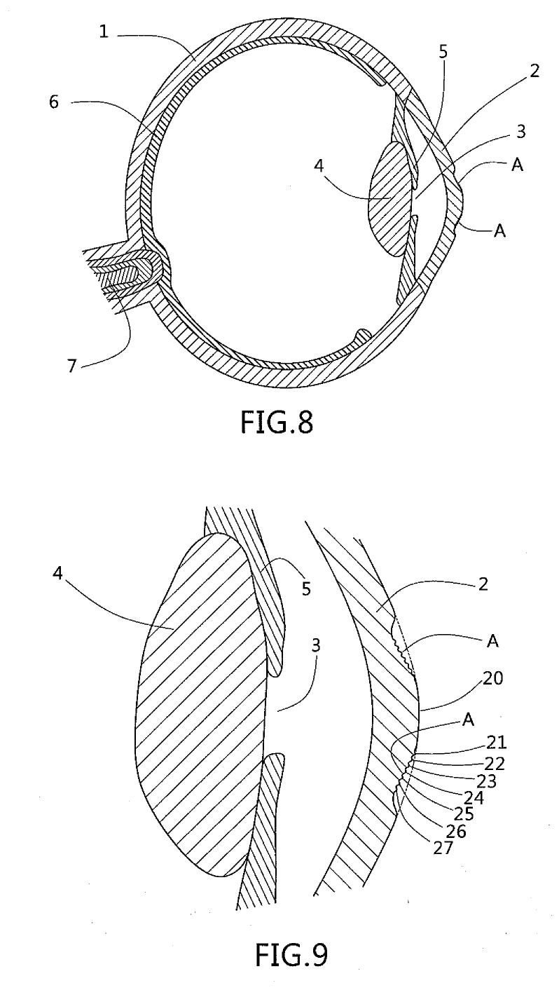

Also, said corrections can be applied to the patient with hyperopia as shown in FIGS. 8 and 9, that is to perform a refractive vision correction 20 with a predetermined curve upon a corresponding area of the center of the cornea 2 when the pupil 3 is in the size of a normal light condition, and then to form a concave surface A by a decided curve outward from the corresponding area of cornea, thereafter, to perform the convex of circular wavy corrections 21, 22, 23 . . . 27 continuously and orderly from inside out upon the concave surface A; each of the corrections would project the images in different positions in front of the retina in order to achieve the goal of correcting presbyopia.

Furthermore, the patient with astigmatism can be under the same way of correction, but the slightly difference is when after performing a refractive vision correction in order to correct astigmatism with a predetermined curve upon a corresponding area of the center of the cornea when the pupil is in the size of a normal light condition, and then to perform other corrections with different predetermined curves outward from the corresponding area of the corneal surface orderly. The corrections can also help to improve vision against presbyopia in the future.

In addition, if the patient is simply with the problem of presbyopia, then can only form a concave surface A in a predetermined area beyond and around the center of the cornea with a determined curve orderly from inside out, and then perform the convex of circular wavy corrections 21, 22, 23 . . . 27 continuously and orderly from inside out upon the concave surface A, in order to prevent presbyopia or improve the vision against presbyopia as well.

1. A refractive vision correction for preventing presbyopia, said correction comprising:
performing the circular wavy corrections orderly from inside out in accordance with different degree of presbyopia upon and beyond a corresponding area of the cornea when the pupil is in a normal light condition.

2. A refractive vision correction as claimed in claim 1, wherein each convex portion of the circular wavy corrections provide correcting to different degrees of presbyopia.

3. A refractive vision correction as claimed in claim 1, wherein the scope of the cornea for performing the preventive and refractive vision correction is the scope beyond the corresponding area of the cornea when the pupil is in a normal light condition and within the area when the pupil is in its most dilation.

4. A refractive vision correction as claimed in claim 1, wherein the cornea surface with performing the circular wavy corrections is an inward concave with a predetermined curve.

5. A refractive vision correction as claimed in claim 1, wherein the corresponding area of the cornea when the pupil is in a normal light condition can be also performed the common refractive vision correction of myopia, hyperopia or astigmatism.
